(19) United States
(12) Patent Application Publication
(10) Pub. No.: US 2024/0425510 A1
(43) Pub. Date: Dec. 26, 2024

(54) PROCESS FOR THE PREPARATION OF ZANUBRUTINIB

(71) Applicant: OLON S.P.A., RODANO (MI) (IT)

(72) Inventors: Alessia MANFREDI, RODANO (MI) (IT); Mattia BERTOLOTTI, RODANO (MI) (IT); Valentina GRANDE, RODANO (MI) (IT); Barbara NOVO, RODANO (MI) (IT)

(73) Assignee: OLON S.P.A., RODANO (MI) (IT)

(21) Appl. No.: 18/697,596

(22) PCT Filed: Oct. 10, 2022
(86) PCT No.: PCT/IB2022/059687

§ 371 Date: Apr. 1, 2024

(30) Foreign Application Priority Data
Oct. 11, 2021 (IT) 102021000025997

(51) Int. Cl. C07D 487/04

(52) U.S. Cl. CPC C07D 487/04

(57) ABSTRACT

It is described a process for the preparation of Zanubrutinib, a drug used for the treatment of adult patients suffering from mantle cell lymphoma (MCL) or Waldenström macroglobulinemia.

### FIELD OF THE INVENTION

The present invention relates to a process for the preparation of Zanubrutinib of formula:

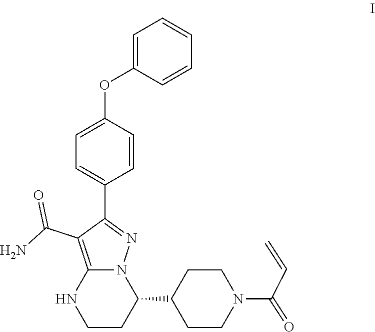 [SMILES: C=CC(=O)N1CCC([C@@H]2CCNc3c(C(N)=O)c(-c4ccc(Oc5ccccc5)cc4)nn32)CC1]

Zanubrutinib is a drug approved in 2019 in the U.S. for the treatment of patients suffering from mantle cell lymphoma (MCL) who have already received at least one previous therapy.

### STATE OF THE ART

The synthesis of Zanubrutinib was described in WO2018033853 and comprises the steps shown in Scheme 1.

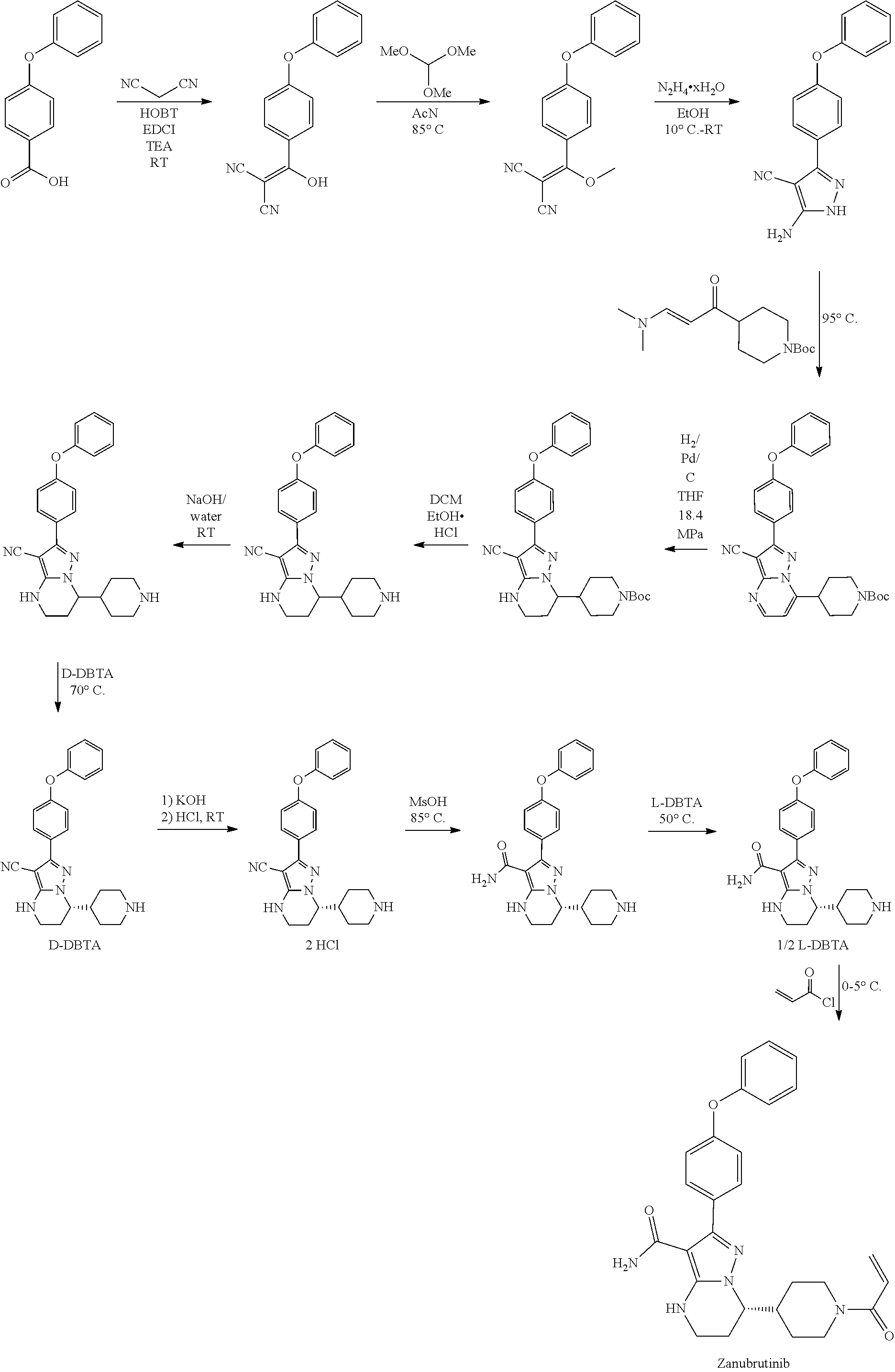

The known process encompasses the formation of the pyrazole-pyrimidine central ring, the reduction of the pyrimidine ring and the subsequent resolution of the thus obtained racemic mixture with D-dibenzoyl tartaric acid.

The compound deriving from said resolution shows an enantiomeric excess of 92%, not compliant with the optical purity specifications required by ICH guidelines on pharmaceutical active ingredients. Therefore, the product needs to be subjected to a subsequent chiral purification with L-dibenzoyl tartaric acid after being hydrolyzed to give the corresponding amide which is in turn acylated to give Zanubrutinib.

The need to carry out multiple chiral resolutions causes a relevant reduction of the process overall yield being about 4%.

### DESCRIPTION OF THE INVENTION

The aim of the present invention is a process for the preparation of Zanubrutinib which is advantageous with respect to the known ones, allowing to eliminate the chiral purification step described in WO2018033853, resulting in an increased yield and a production costs optimization.

Surprisingly, it has been found that, introducing the phenoxy-phenyl group on the pyrazole-pyrimidine ring in the last step by a Suzuki reaction, it is possible to use a single chiral resolution step in the presence of L-dibenzoyl tartaric acid.

In this way, Zanubrutinib is obtained with an overall yield higher than that of the known methods and with a high enantiomeric excess, equal to 99.85% or higher.

The process for the preparation of Zanubrutinib of the invention is shown in the following Scheme 2:

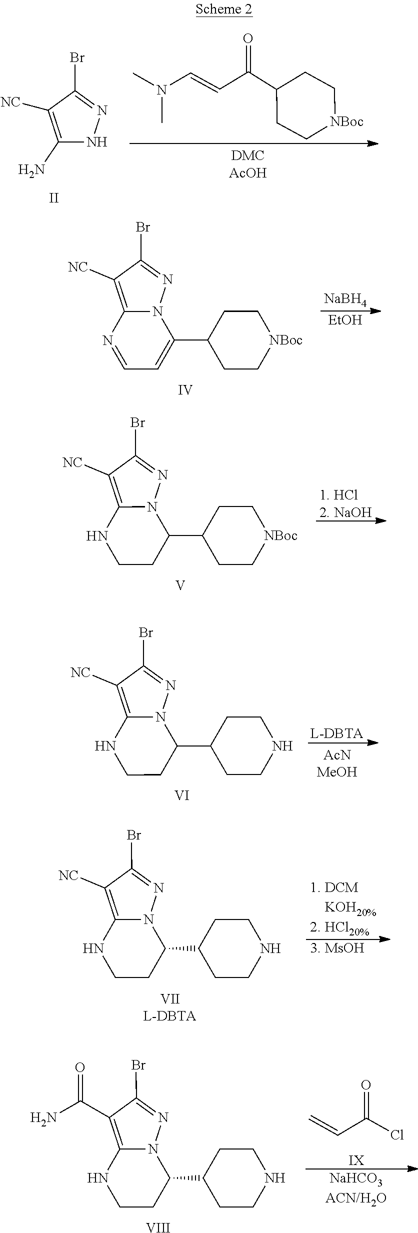

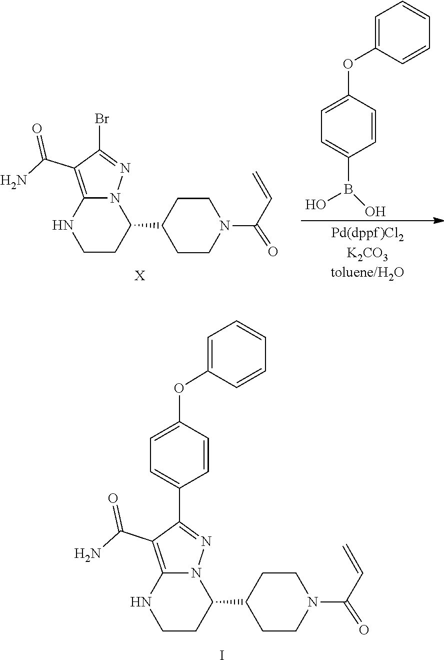

The process for the preparation of Compound of formula (I)

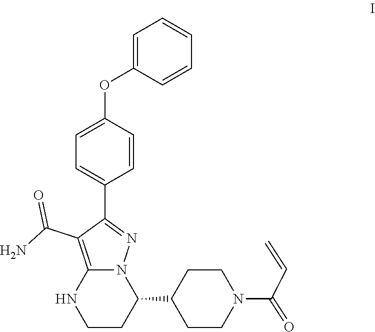 [SMILES: C=CC(=O)N1CCC([C@@H]2CCNc3c(C(N)=O)c(-c4ccc(Oc5ccccc5)cc4)nn32)CC1]

comprises the following steps:
a) reaction of 5-amino-3-bromo-1H-pyrazole-4-carbonitrile (II) with N-Boc-(E)-4-(3-(dimethylamino)acryloyl)piperidine (III) to give (7R,S)-2-bromo-3-cyano-7-(N-Boc-piperidin-4-yl)-4,5,6,7-tetrahydropyrazole[1,5-a]pyrimidine (IV)

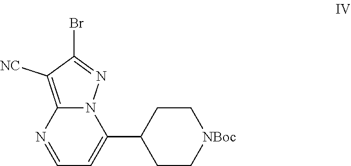 [SMILES: CC(C)(C)OC(=O)N1CCC(c2ccnc3c(C#N)c(Br)nn23)CC1]

b) reduction of (7R,S)-2-bromo-3-cyano-7-(N-Boc-piperidin-4-yl)-pyrazole[1,5-a]pyrimidine (IV) in the presence of sodium borohydride to give (7R,S)-2-bromo-3-cyano-7-(N-Boc-piperidin-4-yl)-4,5,6,7-tetrahydropyrazole[1,5-a]pyrimidine (V)

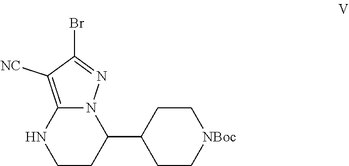 [SMILES: CC(C)(C)OC(=O)N1CCC(C2CCNc3c(C#N)c(Br)nn32)CC1]

c) deprotection of (7R,S)-2-bromo-3-cyano-7-(N-Boc-piperidin-4-yl)-4,5,6,7-tetrahydropyrazole[1,5-a]pyrimidine (V) in acidic condition to give (7R,S)-2-bromo-3-cyano-7-(piperidin-4-yl)-4,5,6,7-tetrahydropyrazole[1,5-a]pyrimidine (VI)

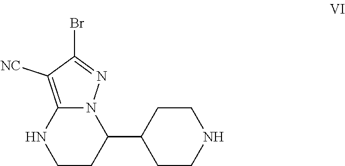 [SMILES: N#Cc1c(Br)nn2c1NCCC2C1CCNCC1]

d) chiral resolution of (7R,S)-2-bromo-3-cyano-7-(piperidin-4-yl)-4,5,6,7-tetrahydropyrazole[1,5-a]pyrimidine (VI) with L-dibenzoyl tartaric acid to give (7S)-2-bromo-3-cyano-7-(piperidin-4-yl)-4,5,6,7-tetrahydropyrazole[1,5-a]pyrimidine (VII)

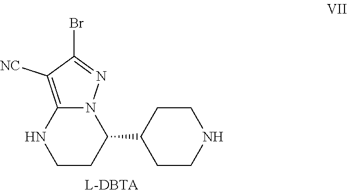 [SMILES: N#Cc1c(Br)nn2c1NCC[C@H]2C1CCNCC1]

e) hydrolysis of (7S)-2-bromo-3-cyano-7-(piperidin-4-yl)-4,5,6,7-tetrahydropyrazole[1,5-a]pyrimidine (VII) to give (7S)-2-bromo-7-(piperidin-4-yl)-4,5,6,7-tetrahydropyrazole[1,5-a]pyrimidin-3-carboxamide (VIII)

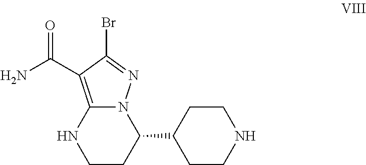 [SMILES: NC(=O)c1c(Br)nn2c1NCC[C@H]2C1CCNCC1]

f) reaction of (7S)-2-bromo-7-(piperidin-4-yl)-4,5,6,7-tetrahydropyrazole[1,5-a]pyrimidine-3-carboxamide (VIII) with an acryloyl halide to give Compound X

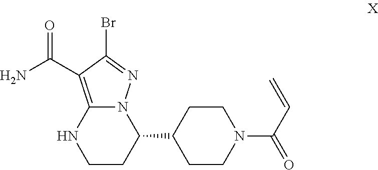 [SMILES: C=CC(=O)N1CCC([C@@H]2CCNc3c(C(N)=O)c(Br)nn32)CC1]

g) Suzuki reaction of Compound X with phenoxyphenyl boronic acid to give Zanubrutinib.

Step a) is conducted in toluene, acetonitrile, dioxane or dichloromethane in the presence of acetic acid at a temperature above 60° C., preferably in dichloromethane, in a ratio dichloromethane to acetic acid of 9 to 1.

Step b) of reduction with sodium borohydride is conducted in a conventional way in alcoholic solvents, preferably ethanol.

Step c) is conducted by treatment with organic or inorganic acids in conventional conditions for the removal of the ter-butoxycarbonyl group, typically by treatment with a HCl solution in ethanol.

Step d) is conducted in a mixture of acetonitrile and methanol in proportions varying from 1:1 to 2:1.

The hydrolysis of the cyano group to carboxyamido group (step e)) can be conducted by treatment with alkaline hydroxides in polar aprotic solvents such as halogenated hydrocarbons, preferably dichloromethane.

Step f) is conducted preferably in a mixture of water and acetonitrile in the presence of bases such as alkaline or alkaline-earth metal carbonates or bicarbonates.

Finally, the Suzuki reaction (step g)) is conducted conventionally in the presence of bases and palladium complexes as catalysts in biphasic systems, for example, toluene/water.

The starting compound 5-amino-3-bromo-1H-pyrazole-4-carbonitrile (II) is commercially available or can be synthesized according to the reaction described in Scheme 3 for reaction of 5-amino-1H-pyrazole-4-carbonitrile with N-bromo-succinimide in dimethylformamide.

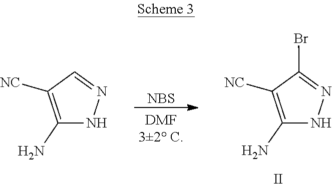

### EXPERIMENTAL

The NMR was measured by a Varian Oxford-300 NMR in deuterated dimethyl sulfoxide (DMSO-d₆) and deuterated chloroform (CDCl₃), using tetramethyl silane (TMS) as internal standard. The chemical shift is available in the unit 10⁻⁶ (ppm).

The MS was measured using a Q-TRAP 3200 (ESI) mass spectrometer of AB-Sciex.

IPC and sample purity were determined by High Performance Liquid Chromatography using a ThermoFischer Vinca DAD and Agilent 1100 DAD (X-Bridge C8, 150×4.6 mm, 3.5 μm) instrument. The chiral measurements were performed using HPLC Agilent 1100 DAD and Waters Alliance (Chiralcel AD-H 250×4.6 mm, 5 μm, Chiralpak IC 250×4.6 mm, 5 μm).

For TLCs, F₂₅₄ Merck silica gel plates with a thickness between 0.15 mm and 0.2 mm are used. For the separation and purification by thin layer chromatography, silica panels with a thickness from 0.4 mm to 0.5 mm are used. The column chromatography uses generally GRACE GmbH 60 RS Å 40-63 μm silica gel as a carrier.

The starting materials can be synthesized according to known methods or can be purchased from Merck KGaA, Aldrich Chemical Company, EOS Med Chem Co., Ltd.

In the Examples of the invention, unless otherwise stated, the reactions were conducted in a temperature range between 20° C. and 30° C.

### Abbreviation List

| | |
|---|---|
| AcN | Acetonitrile |
| AcOH | Acetic acid |
| CY | Cyclohexane |
| D-DBTA | (2S,3S)-dibenzoyl tartaric acid |
| DCM | Dichloromethane |
| DMF | N,N-dimethylformamide |
| DMC | Dimethyl carbonate |
| DSC | Differential Scanning Calorimetry |
| AcOEt | Ethyl acetate |
| EtOH | Ethanol |
| L-DBTA | (2R,3R)-dibenzoyl tartaric acid |
| MeOH | Methanol |
| MS | Mass Spectrometry |
| MsOH | Methane sulfonic acid |
| MTBE | Methyl-t-butyl ether |
| Pd (dppf)Cl₂ | [1,1′-bis(diphenylphosphino)ferrocene]dicloropalladium (II) |
| RT | Room temperature |

### Example 1 Synthesis of Compound IV

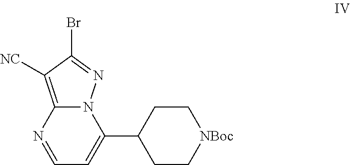 [SMILES: CC(C)(C)OC(=O)N1CCC(c2ccnc3c(C#N)c(Br)nn23)CC1]

In a reaction flask, 5-amino-3-bromo-1H-pyrazole-4-carbonitrile (II) (41 g, 1.0 eq), t-butyl-(E)-4-(3-(dimethylamino)acryloyl)piperidine-1-carboxylate (III) (74.3 g, 1.2 eq), DMC (9 v.) and AcOH (2 v.) were loaded, and heated at 90-95° C. The solution was kept under stirring until the reaction was complete. Then, the solution was cooled down to 20-25° C. and a solid was precipitated. The mixture was filtered, by washing with DMC. The solid was dried under vacuum at 40° C. for 16 hours. 72 g of Intermediate IV were obtained.

¹H NMR (300 MHz, DMSO-d₆) δ 8.82-8.78 (d, J=4.5 Hz, 1H), 7.34-7.32 (d, J=4.5 Hz, 1H), 4.20-4.05 (d, J=12.3 Hz, 2H), 3.70-3.58 (ddd, J=3.9, 12.3, 22.2 Hz, 1H), 3.05-2.70 (m, 2H), 2.10-1.95 (d, J=12.3 Hz, 2H), 1.71-1.41 (ddd, J=3.9, 12.3, 22.2 Hz, 2H), 1.42 (s, 9H).

MS m/z (ESI): 406 [M+1]

### Example 2 Synthesis of Compound V

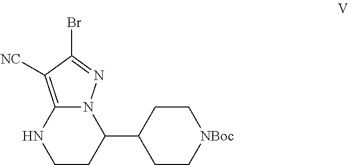 [SMILES: CC(C)(C)OC(=O)N1CCC(C2CCNc3c(C#N)c(Br)nn32)CC1]

A solution of Compound IV (72 g, 1.0 eq) in DCM (5 v.) was added dropwise in 30 minutes in a NaBH₄ suspension (26.8 g, 4.0 eq) in EtOH (5 v.) keeping the temperature below 30° C. The mixture was kept under stirring at room temperature until completion. H₂O (10 v.) was added, and after stirring for 30 minutes, the organic layer was collected and the aqueous layer was extracted with DCM (5 v.). The organic layers were combined and concentrated under vacuum giving 71 g of crude product that was purified by column chromatography: silica gel 40-63 Å, 40 v/v, eluent: 50% v/v CY in AcOEt. The collected solution was concentrated giving 50 g of Intermediate V.

¹H NMR (300 MHz, DMSO-d₆) δ 7.79 (s, 1H), 4.00-3.94 (m, 3H), 3.30-3.10 (m, 2H), 2.80-2.40 (m, 2H), 2.10-1.94 (m, 2H), 1.91-1.80 (m, 1H), 1.59-1.52 (m, 1H), 1.45-1.27 (m, 10H), 1.26-1.07 (m, 2H).

MS m/z (ESI): 410 [M+1]

### Example 3 Synthesis of Compound VI

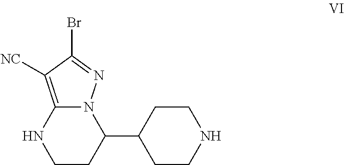 [SMILES: N#Cc1c(Br)nn2c1NCCC2C1CCNCC1]

To a solution of Compound V (50 g, 1.0 eq) in DCM (6 v.), 17% HCl/EtOH (129 g, 5.0 eq) was added in 30 minutes keeping the temperature below 30° C. and left under stirring for 12-18 hours, therefore MTBE (4 v.) was added. The reaction was cooled down to 0-5° C., left under stirring for 1 hour, then it was filtered and the pad was washed with EtOH (1 v.). The collected solid was suspended in a mixture of 50% v/v water in MeOH (16 v.), the suspension was heated up to 60° C. and kept under stirring for 30 minutes. The mass was cooled down slowly to 0-5° C. and a NaOH (2 eq.) in H₂O (0.5 v.) solution was added. The reaction was kept under stirring for 1 hour at 0-5° C. and filtered. The cake was washed with cold water and dried under vacuum at 40-45° C. for 16 hours to give 36.4 g of Intermediate VI.

¹H NMR (300 MHz, DMSO-d₆) δ 7.76 (s, 1H), 4.10-4.00 (br m, 1H), 3.95-3.86 (m, 1H), 3.30-3.10 (m, 1H), 2.80-3.00 (m, 2H), 2.40-2.25 (m, 2H), 2.10-1.80 (m, 4H), 1.50-1.43 (d, J=12.3 Hz, 1H), 1.37-1.31 (d, J=12.3 Hz, 1H), 1.27-1.09 (ddd, J=3.6, 11.76, 22.8 Hz, 2H).

MS m/z (ESI): 310 [M−1]

### Example 4 Synthesis of Compound VII

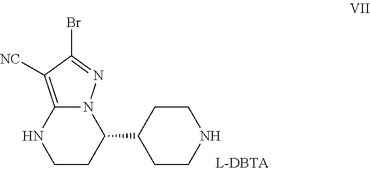 [SMILES: N#Cc1c(Br)nn2c1NCC[C@H]2C1CCNCC1]

In a reaction flask, Compound VI (36.4 g, 1.0 eq) and a mixture of 33% v/v AcN in MeOH (15 v.) were loaded. The suspension was heated up to 60-65° C. and L-DBTA (50.5 g, 1.2 eq) was added. The reaction was kept at 60-65° C. for 16 hours and then cooled down to 20-25° C. stirring for 2 hours at RT. The solid was obtained by filtration, washing the cake with cold water (2 v.), and dried under vacuum at 40° C. for 16 hours to give 33.8 g of Intermediate VII. d.e. 95.80%.

¹H NMR (300 MHz, DMSO-d₆) δ 8.99 (br s, 2H), 7.90-7.98 (m, 4H), 7.84 (s, 1H), 7.58-7.68 (m, 2H), 7.40-7.55 (m, 4H), 5.65 (s, 2H), 3.95-3.86 (dd, J=13.5 Hz, 1H), 3.10-3.30 (m, 4H), 2.65-2.82 (m, 2H), 1.98-2.12 (m, 1H), 1.70-1.95 (m, 2H), 1.60-1.68 (d, J=13.5 Hz, 1H), 1.30-1.55 (m, 3H).

### Example 5 Synthesis of Compound VIII

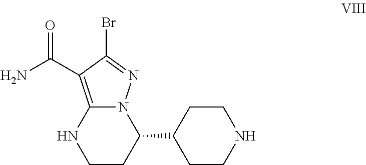 [SMILES: NC(=O)c1c(Br)nn2c1NCC[C@H]2C1CCNCC1]

Under nitrogen atmosphere, a 20% v/v KOH solution with water (3 v.) was added to a suspension of Compound VII (33.8 g, 1.0 eq) in DCM (15 v.). The reaction was kept under stirring for 30 minutes and then the layers were separated. The aqueous layer was extracted with DCM and the combined organic layers were loaded in a reaction flask. 37% HCl (0.36 v.) was added, and the mass was stirred for 30 minutes. The solvent was distilled to a total volume of 135 mL (4 v.). DMC (3 v.) was added, and distilled under vacuum to 135 mL (4 v.). DMC (16 v.) was added, the suspension was kept under vacuum for 1 hour at 20-25° C., then cooled down to 0-5° C. for 1 hour. The solid was filtered, washed with DMC, and dried under vacuum at 40° C. for 16 hours. The solid was loaded in a reaction flask and MsOH (7.0 eq, 2.5 v.) was added. The reaction was kept under stirring at 20-25° C. for 1 hour and then heated up to 85-90° C. until completion. The mass was cooled down slowly at 0-5° C. and H₂O (3 v.) was added dropwise keeping the temperature below 15° C. The mass was stirred for 30 minutes and DCM (3 v.) was added. The organic layer was collected, while the aqueous layer was extracted with DCM (2 v.). The aqueous layer was loaded to a flask wherein DCM (4 v.) was added. While keeping the temperature below 30° C., 20% v/v NaOH was added dropwise until pH 11-12. The organic layer was collected and the aqueous layer was extracted with DCM (3 v.). The organic layers were combined and concentrated under vacuum to yield 15.7 g of Intermediate VIII.

¹H NMR (300 MHz, DMSO-d₆) δ 6.87 (m, 1H), 3.95-3.80 (m, 1H), 3.35-3.15 (m, 2H), 3.10-2.90 (m, 2H), 2.62-2.45 (m, 2H), 2.10-1.70 (m, 3H), 1.65-1.15 (m, 4H).

MS m/z (ESI): 328 [M+1]

### Example 6 Synthesis of Compound X

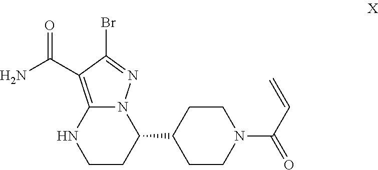 [SMILES: C=CC(=O)N1CCC([C@@H]2CCNc3c(C(N)=O)c(Br)nn32)CC1]

Under nitrogen atmosphere, Compound VIII (15.7 g, 1 eq) was loaded into a mixture of 50% v/v AcN in water and NaHCO₃ (6.0 g, 1.5 eq) was then added. The mass was cooled at 0-5° C. and acryloyl chloride (IX) (4.8 g, 1.1 eq) was added dropwise keeping the temperature below 5° C. The reaction was stirred for 30 minutes at 0-5° C. and then heated up to 20-25° C. AcOEt (10 v.) was added and the layers were separated. The aqueous layer was washed with AcOEt (5 v.) and the organic layers were combined and concentrated under vacuum to give 15 g of Compound X.

¹H NMR (300 MHz, DMSO-d₆) δ 6.88-6.73 (m, 2H), 6.12-6.02 (dd, J=2.4, 7.8 Hz, 1H), 5.68-5.60 (dd, J=2.4, 10.5 Hz, 1H), 4.51-4.46 (m, 1H), 4.17-3.80 (m, 2H), 3.10-2.85 (m, 1H), 2.62-2.40 (m, 1H), 2.28-2.12 (m, 1H), 2.10-1.92 (m, 1H), 1.91-1.80 (m, 1H), 1.69-1.64 (m, 1H), 1.60-1.40 (m, 1H), 1.35-1.05 (m, 3H).

MS m/z (ESI): 382 [M+1]

### Example 7 Synthesis of Zanubrutinib

In a reaction flask under nitrogen Compound X (15.0 g, 1 eq), phenoxyphenyl boronic acid (10.9 g, 1.3 eq), toluene (30 v.) and H₂O (5 v.) were loaded. The mass was stirred for 30 minutes, then Pd(dppf)Cl₂ (3.2 g, 0.1 eq) was added. The reaction was heated up to 90-95° C. and kept under stirring until the reaction was complete, then cooled down to RT, H₂O (15 v.), NaCl (1.5 eq/w) e DCM (40 v.) were added and left under stirring for 30 minutes. The layers were separated and the aqueous layer was washed with DCM. The organic layers were combined and concentrated under vacuum giving 18 g of crude product which was purified by silica gel column: 40-63 Å, 40 v/v, eluent: 3% v/v MeOH in DCM. The collected solution was suspended in AcOEt, filtered and dried under vacuum at 40° C. for 16 hours giving 15 g of Zanubrutinib of formula I.

¹H NMR (300 MHz, DMSO-d₆) δ 7.50-7.47 (d, J=8.7 Hz, 1H), 7.46-7.35 (t, J=7.8 Hz, 1H), 7.18-7.12 (t, J=7.2 Hz, 1H), 7.08-7.00 (t, J=15.3 Hz, 4H), 6.80-6.65 (dd, J=10.5, 16.8 Hz, 1H), 6.67 (s, 1H), 6.10-6.00 (dd, J=2.4, 16.8 Hz, 1H), 5.65-5.55 (dd, J=2.4, 10.5 Hz, 1H), 4.38-4.20 (m, 1H), 4.08-3.92 (m, 2H), 3.35-3.10 (m, 2H), 3.05-2.90 (m, 1H), 2.62-2.40 (m, 1H), 2.30-2.15 (m, 1H), 2.10-1.80 (m, 2H), 1.78-1.65 (m, 1H), 1.61-1.45 (m, 1H), 1.40-1.10 (m, 2H).

MS m/z (ESI): 471 [M+1]

1: A process for the preparation of Zanubrutinib of formula I: 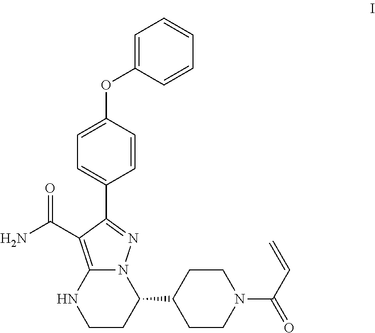 [SMILES: C=CC(=O)N1CCC([C@@H]2CCNc3c(C(N)=O)c(-c4ccc(Oc5ccccc5)cc4)nn32)CC1]
comprising:
a) reacting 5-amino-3-bromo-1H-pyrazole-4-carbonitrile (IT) with N-Boc-(E)-4-(3-(dimethylamino)acryloyl)piperidine (III) to give (7R,S)-2-bromo-3-cyano-7-(N-Boc-piperidin-4-yl)-4,5,6,7-tetrahydropyrazole[1,5-a]pyrimidine (IV) 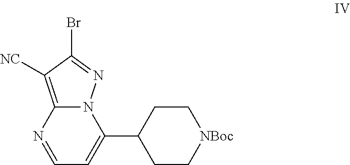 [SMILES: CC(C)(C)OC(=O)N1CCC(c2ccnc3c(C#N)c(Br)nn23)CC1]
b) reducing (7R,S)-2-bromo-3-cyano-7-(N-Boc-piperidin-4-yl)-pyrazole[1,5-a]pyrimidine (IV) in the presence of sodium borohydride to give (7R,S)-2-bromo-3-cyano-7-(N-Boc-piperidin-4-yl)-4,5,6,7-tetrahydropyrazole[1,5-a]pyrimidine (V); 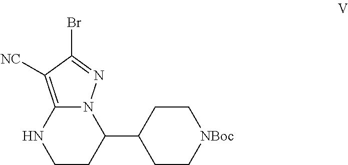 [SMILES: CC(C)(C)OC(=O)N1CCC(C2CCNc3c(C#N)c(Br)nn32)CC1]
c) deprotecting (7R,S)-2-bromo-3-cyano-7-(N-Boc-piperidin-4-yl)-4,5,6,7-tetrahydropyrazole[1,5-a]pyrimidine (V) in an acidic condition to give (7R,S)-2-bromo-3-cyano-7-(piperidin-4-yl)-4,5,6,7-tetrahydropyrazole[1,5-a]pyrimidine (VI); 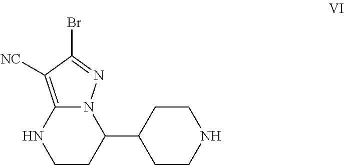 [SMILES: N#Cc1c(Br)nn2c1NCCC2C1CCNCC1]
d) performing chiral resolution of (7R,S)-2-bromo-3-cyano-7-(piperidin-4-yl)-4,5,6,7-tetrahydropyrazole[1,5-a]pyrimidine (VI) with L-dibenzoyltartaric acid to give (7S)-2-bromo-3-cyano-7-(piperidin-4-yl)-4,5,6,7-tetrahydropyrazole[1,5-a]pyrimidine (VII) 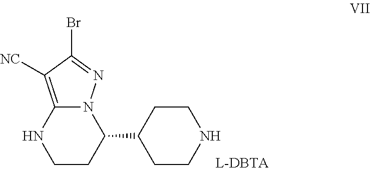 [SMILES: N#Cc1c(Br)nn2c1NCC[C@H]2C1CCNCC1]
e) hydrolyzing (7S)-2-bromo-3-cyano-7-(piperidin-4-yl)-4,5,6,7-tetrahydropyrazole[1,5-a]pyrimidine (VII) to give (7S)-2-bromo-7-(piperidin-4-yl)-4,5,6,7-tetrahydropyrazole[1,5-a]pyrimidin-3-carboxamide (VIII) 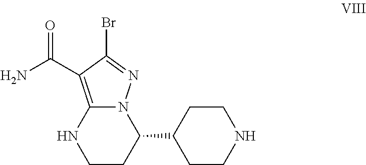 [SMILES: NC(=O)c1c(Br)nn2c1NCC[C@H]2C1CCNCC1]
f) reacting (7S)-2-bromo-7-(piperidin-4-yl)-4,5,6,7-tetrahydropyrazole[1,5-a]pyrimidine-3-carboxamide (VIII) with an acryloyl halide to give Compound X 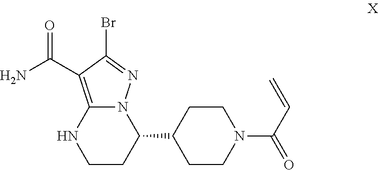 [SMILES: C=CC(=O)N1CCC([C@@H]2CCNc3c(C(N)=O)c(Br)nn32)CC1]
g) performing a Suzuki reaction of Compound X with phenoxyphenyl boronic acid to give Zanubrutinib.

2: The process according to claim 1 wherein:
the reaction a) is conducted at a temperature greater than 60° C.; and
the reaction a) is conducted in toluene, acetonitrile, dioxane, or dichloromethane.

3: The process according to claim 1 wherein the reaction a) is conducted in dichloromethane.

4: The process according to claim 1, wherein:
the reaction a) is conducted in dichloromethane and acetic acid; and
a volume ratio of dichloromethane to acetic acid is 9:2.

5: The process according to claim 1 wherein:
the chiral resolution d) is conducted in acetonitrile and methanol; and
a volume ratio of acetonitrile to methanol is 1:1 to 2:1.
